(19) United States
(12) Patent Application Publication
(10) Pub. No.: US 2020/0078775 A1
(43) Pub. Date: Mar. 12, 2020

(54) REGENERATION OF DEHYDROAROMATIZATION OF CATALYST WITH CONSTANT CONVERSION

(71) Applicant: SABIC Global Technologies B.V., Bergen op Zoom (NL)

(72) Inventors: Amit Kumar, Bangalore (IN); Sreenivasarao Gajula, Bangalore (IN); Ziyad Kottavarithottil, Bangalore (IN); Eswara Rao Mupparaju, Bangalore (IN)

(21) Appl. No.: 16/567,063

(22) Filed: Sep. 11, 2019

Related U.S. Application Data
(60) Provisional application No. 62/730,291, filed on Sep. 12, 2018.

(51) Int. Cl. B01J 29/48; B01J 29/90; B01J 38/10; C07C 2/76

(52) U.S. Cl. CPC B01J 29/48; B01J 29/90; C07C 2529/48; C07C 2/76; B01J 38/10

(57) ABSTRACT

A method for dehydroaromatization of methane, comprising: introducing methane and carbon dioxide into an aromatization reactor; converting a portion of the methane to aromatic hydrocarbons in the presence of a dehydroaromatization catalyst at a temperature of 700 to 850° C.; regenerating the dehydroaromatization catalyst with hydrogen at a regeneration temperature of 700 to 800° C.; and repeating the introducing, the converting and the regenerating; wherein after every 2 to 5 cycles of the regenerating, increasing the regeneration temperature by 5 to 15° C.

### BACKGROUND

The present application relates to methods of using dehydroaromatization catalysts for the conversion of methane to aromatic products.

Aromatic hydrocarbons, for example, benzene, toluene, ethylbenzene, xylene, and polyaromatic hydrocarbons such as naphthalene, are important commodity chemicals in the petrochemical industry.

A method of preparing aromatic hydrocarbons is by the dehydroaromatization (DHA) of methane, as methane is one of the most abundant organic compounds on Earth. For example, methane is the major constituent of natural gas; large amounts of methane are trapped in marine sediments as hydrates and in coal shale as coal bed methane; and it can also be derived from a biomass such as a biogas.

The dehydroaromatization of methane is becoming increasingly important. Methane dehydroaromatization over catalysts is a promising process for the production of valuable aromatic compounds and hydrogen from natural gas.

Catalyst deactivation due to coke formation is one of the main drawbacks of the dehydroaromatization of methane. Coke formation can decrease the life of the catalyst. The equilibrium of the dehydroaromatization of methane is also low due to coke formation on the catalyst. Coke formation is largely favored at high temperatures, particularly at 700° C. and above. Coke formation can be of two types, hard coke and soft coke. Hard coke is mainly known as graphitic type coke. Soft coke can be polyaromatic deposits.

US 2013/0090506 to Ogawa is directed to a method for producing aromatic hydrocarbon. When producing an aromatic hydrocarbon by a contact reaction of a lower hydrocarbon with a catalyst, the aromatic hydrocarbon is produced stably for a long time while maintaining a high aromatic hydrocarbon yield. The process can include a regeneration step for regenerating the catalyst, and the processes can be repeated.

CN 104326854 to Cheng et al. is directed to a methane oxygen-free aromatization reaction technology with catalyst pre-carbonization. With this technology, a catalyst pre-carbonization connection apparatus is arranged in a regeneration continuous circulation reaction system in a methane-to-aromatic hydrocarbon reaction.

US 2013/0012747 to Ma et al. is directed to a method of manufacture of an aromatic compound. The process includes a reaction process of initiating the contact reaction between the lower hydrocarbon and the catalyst thereby obtaining the aromatic hydrocarbons and hydrogen, and a regeneration process of regenerating the catalytic activity by bringing hydrogen into contact with the catalyst used in the reaction process. The reaction process and the regeneration process are repeated thereby producing the aromatic hydrocarbons and hydrogen. In the reaction process, carbon monoxide is added to the lower hydrocarbons. It is preferable that the reaction temperature is no lower than 820° C.

U.S. Pat. No. 6,239,057 to Ichikawa et al. is directed to a catalyst for the conversion of low carbon number aliphatic hydrocarbons to higher carbon number hydrocarbons. A catalyst for producing higher carbon number hydrocarbons, e.g., benzene from low carbon number hydrocarbons such as methane has been developed. The catalyst comprises a porous support such as ZSM-5 which has dispersed thereon rhenium and a promoter metal such as iron, cobalt, vanadium, manganese, molybdenum, tungsten and mixtures thereof. A process for preparing the catalyst and a process for converting low carbon number aliphatic hydrocarbons to higher number hydrocarbons in the presence of CO or CO₂.

Hard coke can be removed under dilute oxygen at lower temperatures (400 to 550° C.), while soft coke can be removed by pure hydrogen at higher temperatures (700 to 850° C.). As described herein, the term “coke” is used to mean carbon containing solid materials, which are essentially non-volatile solids at the reaction conditions.

### SUMMARY

Disclosed herein is a method for dehydroaromatization of methane.

A method for dehydroaromatization of methane, comprising: introducing a feed stream of methane and carbon dioxide to an aromatization reactor; converting a portion of the methane to aromatic hydrocarbons in the presence of a dehydroaromatization catalyst, preferably Mo-HZSM-5 catalyst; regenerating the dehydroaromatization catalyst with hydrogen at a regeneration temperature of 700 to 800° C.; repeating the introducing, the converting and the regenerating; and periodically increasing the regeneration temperature by 5 to 15° C.

### BRIEF DESCRIPTION OF THE FIGURES

FIG. 1 is a graph of methane conversion over time according to the processes disclosed herein.

FIG. 2 is a graph of methane conversion over time according to the processes disclosed herein as compared with a process carried out at a constant temperature.

### DETAILED DESCRIPTION

This invention addresses the problem of reduced catalyst activity in a methane dehydroaromatization reaction due to coke formation. Disclosed herein is the usage of hydrogen to de-coke the catalyst and regain catalyst activity. It has been discovered herein that regeneration of a coked catalyst with hydrogen containing stream (e.g., comprising at least 100% by volume (vol %) hydrogen), wherein after several regeneration cycles, the temperature of the reaction (and the regeneration) is increased, will improve the overall process and feasibility of catalyst regeneration. For example, the performance is improved when the dehydroaromatization of methane is carried out until a percent conversion of the methane decreases by, for example 10 to 15% from an initial value. Once the conversion has decreased, the flow of methane and CO₂ is stopped, and the catalyst is regenerated with hydrogen. After a predetermined number of regeneration cycles or after a decrease in conversion percentage, the reaction temperature is increased. For example, after a decrease in conversion percentage of 5 to 20%, preferably 8 to 15%, or 10 to 15%, the reaction temperature is increased, or after 2 to 8, preferably 2 to 5, regeneration cycles, the reaction temperature is increased. In other words, after a decrease of a conversion percentage from an initial percentage; and subsequently, a decrease in a conversion percentage from the conversion percentage attained after the increase in regeneration temperature. This process can be continued until the temperature of 800° C. is reached. Above 800° C., graphitic coke can form.

The methane dehydroaromatization reaction comprises contacting a dehydroaromatization catalyst with a methane feed at an initial reaction temperature of 700-780° C. The methane is co-fed to the catalyst with carbon dioxide and is converted to aromatic compound(s). The feed stream can comprise 85 to 100 vol %, preferably 90 to 100 vol %, or 90 to 96 vol % methane, based upon the total volume of the feed stream. The feed stream can comprise 0 to 10 vol % (e.g., greater than 0 to 10 vol %), preferably 5 to 10 vol %, or 0 to 4 vol % (e.g., greater than 0 to 4 vol %) carbon dioxide, based upon the total volume of the feed stream. After a set period of time or after a decrease in the conversion percentage, the catalyst is regenerated. The period of time can be up to 120 minutes (min), e.g., 15 min to 60 min, or 15 min to 30 min. The decrease in conversion percentage can be a decrease from an initial conversion percentage of greater than or equal to 15%, e.g., a decrease of 10 to 20% or 10 to 15%.

Regeneration can be carried out, e.g., at the same temperature as the dehydroaromatization reaction. Hydrogen is introduced to the catalyst to react with and remove the coke from the catalyst. The regeneration can also be for a set period of time or can be based upon a measurement of the effluent stream from the reactor. The period of time can be up to 120 min, e.g., 30 min to 90 minutes, or 30 min to 60 minutes, or 45 to 60 minutes. If the cycle time is based upon measurement of the effluent stream, regeneration can be stopped when the amount of methane is less than or equal to 0.2%, e.g., a decrease of 0 to 1% or 0 to 0.2%.

The hydrogen stream can comprise greater than or equal to 90 vol %, preferably greater than or equal to 95 vol %, or greater than or equal to 99 vol % hydrogen, or pure hydrogen (for example 100 vol % hydrogen). The hydrogen stream can be added directly to the methane dehydroaromatization reactor for the first regeneration of the catalyst. The hydrogen stream can be introduced at a GHSV of greater than 1,000 ml·g⁻¹ h⁻¹, preferably 1,000 to 4,000 ml·g⁻¹ h⁻¹, or 2,500 to 3,500 ml·g⁻¹ h⁻¹.

Optionally, the hydrogen gas can be supplied from the product stream of the methane dehydroaromatization reaction. Particularly, the product stream of the methane dehydroaromatization reaction can be processed further to separate pure hydrogen from the remainder of the products. The hydrogen separated from the product stream can be greater than 90 vol % hydrogen. The hydrogen separated from the product stream with no more than 0.1 to 3% residual methane of the methane dehydroaromatization reaction can be used as the hydrogen stream for catalyst regeneration to save cost of supplying an additional hydrogen stream.

After the regeneration, another cycle of methane dehydroaromatization can be performed by ceasing introduction of the hydrogen stream to the reactor and reintroducing the feed stream. After 2 to 8 regeneration cycles, the reaction temperature can be increased. For example, the reaction temperature can be increased after greater than or equal to a 10% drop (preferably greater than or equal to 12% drop or greater than or equal to 15% drop) in catalyst activity followed by hydrogen regeneration and at the start of dehydroaromatization reaction. The temperature increase can be 5 to 20° C., preferably 5 to 15° C., or 8 to 12° C.

For example, a first, second, and third cycle can comprise dehydroaromatization for 15 to 60 minutes at a pressure of 5 to 100 MPa, and a reaction temperature of 750° C., followed by regeneration for a period of 30 to 90 minutes at 750° C. Fourth, fifth, sixth, and seventh cycles can comprise dehydroaromatization for 15 to 60 minutes at a pressure of 5 to 100 MPa, and a reaction temperature of 760° C., followed by regeneration for a period of 30 to 90 minutes at 760° C. Eighth twelfth cycles can comprise dehydroaromatization for 15 to 60 minutes at a pressure of 5 to 100 MPa, and a reaction temperature of 770° C., followed by regeneration for a period of 30 to 90 minutes at 770° C. Thirteenth and Fourteenth cycles can comprise dehydroaromatization for 15 to 60 minutes at a pressure of 5 to 100 MPa, and a reaction temperature of 780° C., followed by regeneration for a period of 30 to 90 minutes at 780° C. This can be continued until a temperature of 800° C. or more has been met. Preferably is it continued up to a temperature of 800° C. since graphitic coke can form at temperatures exceeding 800° C.

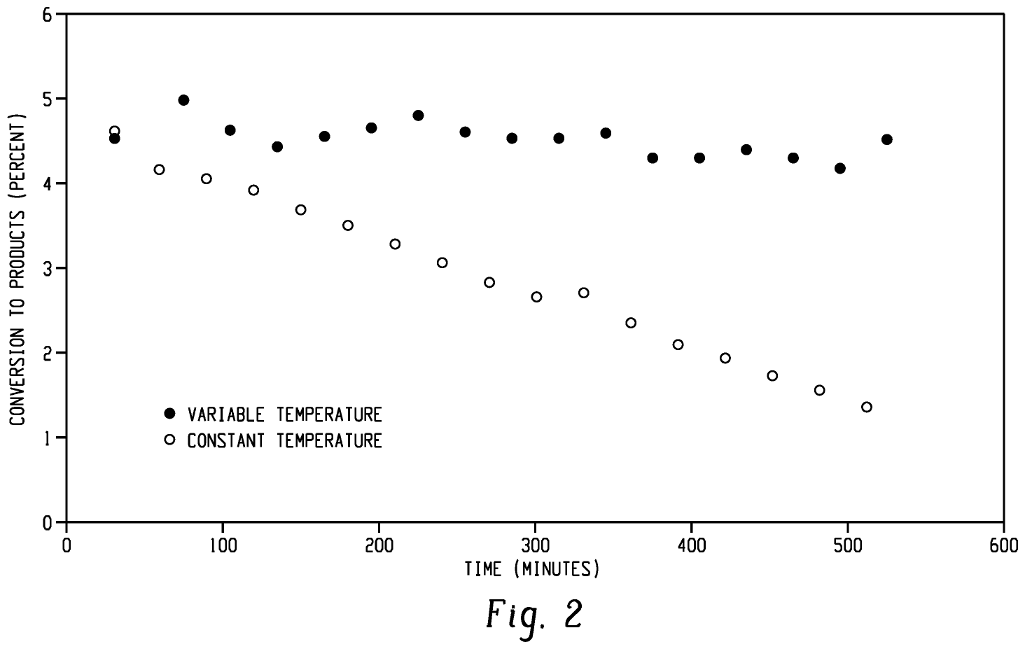

Increasing the reaction temperature has been found to increase the catalyst conversion efficiency by maintaining the percent conversion for a longer period of time. From FIG. 2 and the reaction data, it is believed that there will be a large (e.g., 5 fold) increase in catalyst stability.

The aromatic compound stream produced by the dehydroaromatization reaction comprises at least one of benzene, toluene, naphthalene, or xylene. The aromatic compound can comprise at least one of benzene or toluene. The aromatic compound stream can further comprise traces, e.g., less than 5 vol % (e.g., 1 to 5 vol %) total, of one or more of ethane, ethylene, propene, or propane. Desirably, the aromatic compound stream substantially comprises benzene (e.g., greater than or equal to 80 vol % of the aromatic compound can be benzene).

The aromatic compound(s) can be produced under reaction conditions including a temperature of 700 to 800° C., preferably 750 to 800° C. The pressure can be 5 megaPascal (MPa) to 150 MPa, preferably 30 MPa to 100 MPa, or 40 MPa to 60 MPa. The production rate can be a gas hourly space velocity (GHSV) of greater than or equal to 250 milliliters per grams per hour (ml g⁻¹ h⁻¹), preferably 250 to 350 ml·g⁻¹ h⁻¹, or 275 to 325 ml·g⁻¹ h⁻¹.

A zeolite catalyst can be used in a methane dehydroaromatization reaction. The aromatization catalyst can include the catalytic metal in an amount in a range of 2 weight % (wt %) to 7 wt %, or 3 wt % to 6 wt %, based on the weight of the inorganic support. The catalytic metal can include at least one of chromium, cobalt, gallium, iron, magnesium, molybdenum, vanadium, or zinc, preferably the catalyst comprises molybdenum. Desirably, the aromatization catalyst includes a catalytic metal on an inorganic support. The inorganic support can be an inorganic oxide such as zeolite, preferably a zeolite in the hydrogen form. The zeolite can be at least one of a zeolite Y, zeolite X, mordenite, ZSM-5 (such as HZSM-5), ALPO-5, VPI-5, FSM-16, MCM-22, or MCM-41. In aspects, the zeolite may be MCM-22. Desirably, the zeolite comprises HZSM-5. The zeolite can have a silica to alumina molar ratio in a range of 10 to 50, or 13 to 30, preferably 25 to 30. A zeolite catalyst can be, for example a Mo/ZSM-5, Mo/ZSM-11 and Mo/MCM22. For example, a ZSM-5 catalyst can be used in a methane dehydroaromatization reaction, e.g., a molybdenum oxide ZSM-5 (Mo-oxide/ZSM-5) catalyst. A Mo-oxide/ZSM-5 catalyst provides good catalytic performance, a high aromatic compound selectivity and productivity (for example greater than 85%, and even as high as 90% selectivity).

This disclosure is further illustrated by the following examples, which are exemplary and non-limiting.

### EXAMPLES

A methane to benzene reaction was carried out in a fixed bed reactor using a Mo/HZSM-5 catalyst, charged into an Inconel reactor.

An aromatization catalyst, i.e., molybdenum/H-ZSM-5 catalyst, was prepared using the following steps. The pH of an ammonium hepta molybdate solution (5 wt % concentration) was adjusted with a dropwise addition of an aqueous ammonia solution (25 wt % concentration) until a pH of 10 was reached. Then, an H-ZSM-5 zeolite catalyst support was contacted with the ammonium hepta molybdate solution for 2 hours to allow the molybdenum to penetrate the pores and react with the active centers of the H-ZSM-5 zeolite. The aromatization catalyst was dried at a temperature of 120° C. in an air oven for 12 hours. The aromatization catalyst was then calcined by heating the aromatization catalyst starting from room temperature to 550° C. at a rate of 2.5° C. per minute in a calcination furnace using flowing air (20 to 30° C., 5-30% relative humidity, 0.1 to 1 milliliters per second (ml/s) velocity) for 16 hours. After calcination the aromatization catalyst, a Mo/HZSM-5 catalyst, was cooled to room temperature in the flowing air (20 to 30° C., 5 to 30 relative humidity), at a flow rate of 0.1 to 1 ml/s.

The performance of the Mo/HZSM-5 catalyst was studied at a temperature in the range of 750 to 800° C. at a gas hourly space velocity of 12,500 ml·g⁻¹ h⁻¹. The catalyst was first carburized using 20 wt % methane and 80 wt % hydrogen from room temperature to 650° C. at constant pressure of 5 atm. The catalyst was pre-reduced in-situ by molybdenum oxide carburization with a feed mixture of CH₄ (20 vol %)+H₂ (80 vol %) from room temperature to 650° C. at heating rates of 2° C./min and 10° C./min at a flow rate equivalent to a GHSV of 7,500 mlg⁻¹ hr⁻¹ and then the reaction was started.

Reaction and regeneration for 17 cycles were carried out in a fixed bed reactor on Mo/HZSM-5 catalyst, charged into an Inconel reactor. The performance of the catalyst was studied at temperatures from 750 to 800° C. at a gas hourly space velocity (GHSV) of the feed of 12,500 mlg⁻¹ hr⁻¹. The reaction was carried out under 98 vol % CH₄ and 0.71 vol % CO₂ for 30 minutes, followed by 60 minutes of hydrogen regeneration at a temperature 750 to 800° C. with a stream comprising 100 vol % hydrogen. The major products of the methane to benzene reaction are benzene, toluene, naphthalene and coke. Coke formation on the surface of the catalyst can block the pores which results in the deactivation of the catalyst. The coke was removed from the surface of the catalyst using the hydrogen regeneration.

The reaction temperature was not isothermal as the temperatures were changed in steps of 10° C. after every 4 cycles throughout the 17 cycles to maintain the constant conversion. The reaction temperature was 750° C. at the start of the reaction. After every four cycles, the conversion decreased by 10-12%. In order to maintain conversion, the reaction and regeneration temperature was increased by 10° C.

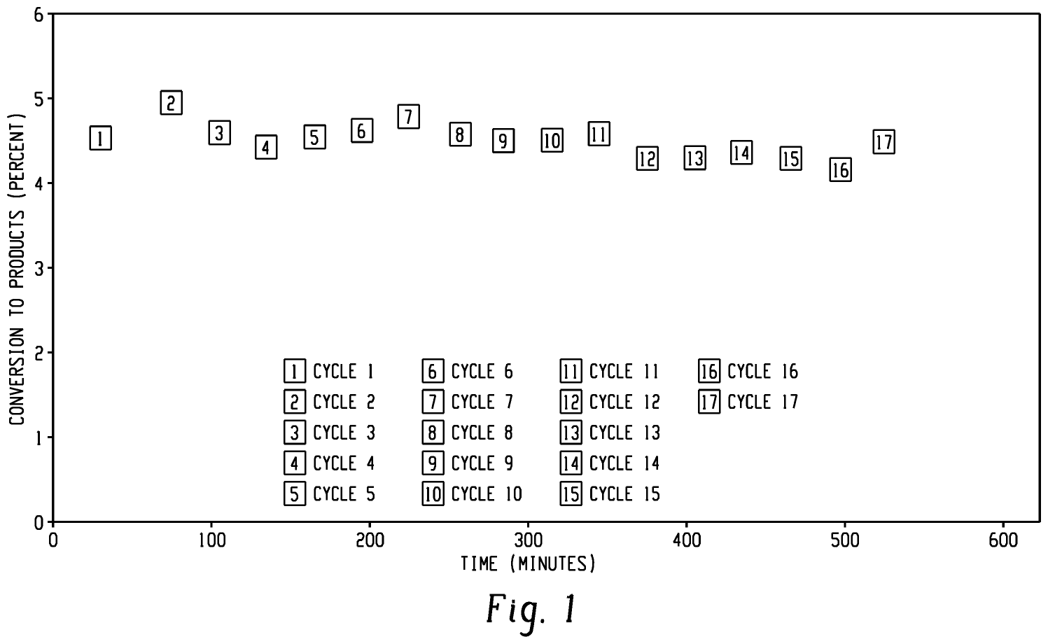

The data shown in FIG. 1 indicates that stable conversion can be maintained throughout the 17 cycles by changing the temperature in steps. Each point on FIG. 1 represents 30 minutes of reaction time. The benzene yields during these 17 cycles were stable, which shows that the catalyst was active during all cycles. Benzene selectivity during all 17 cycles was found to be between 4 and 5% as shown in FIG. 1. During cycles 1-4, the temperature was maintained at 750° C., during cycles 4-8 the temperature was maintained at 760° C., during cycles 8-12 the temperature was maintained at 770° C., during cycles 12-16 the temperature was maintained at 780° C., during cycle 17 the temperature was maintained at 790° C.

The data shown in FIG. 2 compares the temperature adjustment described above to a constant temperature process. As is illustrated, stable conversion cannot be maintained without the temperature adjustment. The variable temperature example is the same as for FIG. 1. The constant temperature data was obtained by reaction and regeneration for 17 cycles in the same Inconel fixed bed reactor using the aromatization catalyst produced above. The performance of the catalyst was studied at a temperature of 750 to 790° C. at a GHSV of the feed of 12,500 mlg⁻¹ hr⁻¹. The reaction was carried out under 98 vol % CH₄ and 0.71 vol % CO₂ for 30 minutes, followed by 60 minutes of hydrogen regeneration at a temperature 750 to 790° C. with a stream comprising 100 vol % hydrogen. Each point on FIG. 2 represents 30 minutes of reaction time. The benzene yields during the cycles where the temperature was not maintained decreased significantly after each cycle.

Set forth below are some of the aspects of the methods disclosed herein.

Aspect 1: A method for dehydroaromatization of methane, comprising: introducing a feed stream of methane and carbon dioxide to an aromatization reactor; converting a portion of the methane to aromatic hydrocarbons in the presence of a dehydroaromatization catalyst, preferably Mo-HZSM-5 catalyst; regenerating the dehydroaromatization catalyst with hydrogen at a regeneration temperature of 700 to 800° C.; repeating the introducing, the converting and the regenerating; and periodically increasing the regeneration temperature by 5 to 15° C.

Aspect 2: The method of Aspect 1, wherein the catalyst is regenerated only with hydrogen.

Aspect 3: The method of any of the preceding aspects, wherein the reaction temperature is increased after a decrease in conversion percentage of 5 to 20%, preferably 8 to 15%, or 10 to 15%.

Aspect 4: The method of any of the preceding aspects, wherein the reaction temperature is increased after every 2 to 8 cycles, preferably after 2 to 5 cycles, or 3 to 5 cycles, of the regenerating.

Aspect 5: The method of any of the preceding aspects, wherein the converting is initially at a temperature of 700 to 780° C., preferably 730 to 770° C., or 745 to 755° C.

Aspect 6: The method of any of the preceding aspects, wherein the feed stream comprises greater than 90 vol % methane and 0.05 to 3 vol % (preferably 0.05 to 2 vol %) carbon dioxide, based upon a 100 vol % of the feed stream.

Aspect 7: The method of any of the preceding aspects, wherein the aromatization reactor is a fixed bed reactor.

Aspect 8: The method of any of the preceding aspects, wherein the dehydroaromatization catalyst is carburized before being used in the aromatization reactor.

Aspect 9: The method of any of the preceding aspects, wherein the introducing of the methane and carbon dioxide is at a gas hourly space velocity of greater than or equal to 1,000 ml·g⁻¹ h⁻¹.

Aspect 10: The method of any of the preceding aspects, wherein the dehydroaromatization catalyst is carburized before being used in the aromatization reactor; wherein the reaction temperature is increased after a decrease in conversion percentage of greater than or equal to 5%, preferably greater than or equal to 8%; wherein the converting is initially at a temperature of greater than or equal to 745° C.; and wherein the feed stream comprises 0.05 to 3 vol %, based upon a 100 vol % of the feed stream.

Aspect 11: The method of Aspect 10, wherein the feed stream comprises 0.05 to 2 vol % carbon dioxide, based upon a 100 vol % of the feed stream.

Aspect 12: A method for dehydroaromatization of methane, comprising: introducing a feed stream of methane and carbon dioxide to an aromatization reactor; converting a portion of the methane to aromatic hydrocarbons in the presence of a dehydroaromatization catalyst, preferably Mo-HZSM-5 catalyst; regenerating the dehydroaromatization catalyst with hydrogen at a regeneration temperature of 750 to 810° C. (preferably 750 to 800° C.); repeating the introducing, the converting and the regenerating; and increasing the regeneration temperature by 8 to 12° C. after a decrease in conversion percentage of 5 to 20%, preferably 8 to 15%, or 10 to 15% (e.g., a decrease of a conversion percentage from an initial percentage; and subsequently, decrease in a conversion percentage from the conversion percentage attained after the increase in regeneration temperature).

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for dehydroaromatization of methane, comprising:
introducing a feed stream of methane and carbon dioxide to an aromatization reactor;
converting a portion of the methane to aromatic hydrocarbons in the presence of a dehydroaromatization catalyst, preferably Mo-HZSM-5 catalyst;
regenerating the dehydroaromatization catalyst with hydrogen at a regeneration temperature of 700 to 800° C.;
repeating the introducing, the converting and the regenerating; and
periodically increasing the regeneration temperature by 5 to 15° C.

2. The method of claim 1, wherein the catalyst is regenerated only with hydrogen.

3. The method of claim 1, wherein the reaction temperature is increased after a decrease in conversion percentage of 5 to 20.

4. The method of claim 1, wherein the reaction temperature is increased after every 2 to 8 cycles of the regenerating.

5. The method of claim 1, wherein the converting is initially at a temperature of 700 to 780° C.

6. The method of claim 1, wherein the feed stream comprises greater than 90 vol % methane and 0.05 to 3 vol % carbon dioxide, based upon a 100 vol % of the feed stream.

7. The method of 1, wherein the aromatization reactor is a fixed bed reactor.

8. The method of any claim 1,
wherein the dehydroaromatization catalyst is carburized before being used in the aromatization reactor;
wherein the reaction temperature is increased after a decrease in conversion percentage of greater than or equal to 5%;
wherein the converting is initially at a temperature of greater than or equal to 745° C.; and
wherein the feed stream comprises 0.05 to 3 vol % carbon dioxide, based upon a 100 vol % of the feed stream.

9. The method of claim 8, wherein the feed stream comprises 0.05 to 2 vol % carbon dioxide, based upon a 100 vol % of the feed stream.

10. The method of claim 3 wherein the reaction temperature is increased after a decrease in conversion percentage of 8 to 15%.

11. The method of claim 4 wherein the reaction temperature is increase after every 3 to 5 cycles.

12. The method of claim 5 wherein the converting is initially at a temperature of 730 to 770° C.

13. The method of claim 6 wherein the feed stream comprises 0.05 to 2 vol % carbon dioxide based upon 100 vol % of the feed stream.
